(19) United States
(12) Patent Application Publication
(10) Pub. No.: US 2014/0153805 A1
(43) Pub. Date: Jun. 5, 2014

(54) METHOD AND HYBRID IMAGING MODALITY FOR PRODUCING A COMBINATION IMAGE

(71) Applicant: SIEMENS AKTIENGESELLSCHAFT, Munich (DE)

(72) Inventor: Sebastian SCHMIDT, Weisendorf (DE)

(73) Assignee: Siemens Aktiengesellschaft, Munich (DE)

(21) Appl. No.: 14/087,074

(22) Filed: Nov. 22, 2013

(30) Foreign Application Priority Data
Dec. 4, 2012 (DE) 102012222201.4

(51) Int. Cl. G06T 11/00; G01R 33/48; A61B 5/055; A61B 6/03; A61B 6/00

(52) U.S. Cl. CPC G06T 11/008; A61B 6/037; A61B 6/4417; A61B 6/507; A61B 6/5247; A61B 6/032; A61B 5/055; G01R 33/481
USPC 382/131

(57) ABSTRACT

A method is disclosed for producing a combination image. In an embodiment, the method includes acquiring at least one PET image data set depicting a region of interest, in particular at least part of a lung; acquiring at least one perfusion image data set depicting the region of interest using a second imaging modality; establishing a threshold value for the perfusion image data set and selecting the regions of the perfusion image data set that are below the threshold value and/or inverting the color palette or grayscale palette of the perfusion image data set; and combining the PET image data set and the perfusion image data set to form a combination image. A hybrid imaging modality is also disclosed.

### PRIORITY STATEMENT

The present application hereby claims priority under 35 U.S.C. §119 to German patent application number DE 102012222201.4 filed Dec. 4, 2012, the entire contents of which are hereby incorporated herein by reference.

### FIELD

At least one embodiment of the present invention generally relates to a method and a hybrid imaging modality for producing a combination image.

### BACKGROUND

Positron emission tomography, or PET for short, is an imaging method which enables the distribution of a radioactive substance in an object under examination to be displayed. Positron-emitting radionuclides are used in PET, wherein a detector ring is disposed around the object under examination to acquire the scan data. When an emitted positron is annihilated with an electron, two photons are released which move apart in opposite directions. If two photons are detected with the detector ring in a predefined time segment, this is deemed to be a coincidence and therefore an annihilation event.

A single annihilation event still gives no indication of a spatial distribution. It is only by recording a plurality of annihilation events that a PET image data set can be calculated from the individual “line of response”, or LOR for short. In the following, the acquisition of a PET image data set will be understood as meaning the spatially resolved recording of annihilation events with subsequent calculation of the PET image data set.

The scanning time varies depending on the radioactivity of the radionuclide and the desired signal intensity, but is approximately at least one minute.

The passage of a radionuclide through the body merely provides information about the distribution paths of the radionuclide itself. In order to provide information about metabolic processes it is known to provide metabolites with a radionuclide, wherein the organism ideally makes no distinction between the original metabolite and the metabolite provided with a radionuclide. Such substances are also known as radiopharmaceuticals.

A well known and frequently used radiopharmaceutical is fluorodeoxyglucose (18F-FDG) which is metabolized instead of glucose. Tumor cells have a higher metabolic rate than normal cells, with FDG also being taken up and FDG-6-phosphate metabolized. As no further changes take place subsequently, FDG accumulates in the tumor cells.

However, in addition to tumor cells, accumulation also takes place in other glucose-processing tissues. This is normally uncritical, as these regions of the body or more specifically tissues are well known and can be identified in a PET image data set.

The paper Kamel E. M. et al., Occult lung infarction may induce false interpretation of 18F-FDG PET in primary staging of pulmonary malignancies, Eur J Nucl Med Mol Imaging, 32: 641-646, 2005 shows that FDG accumulation also takes place in microinfarctions in the lung and not only in the case of pulmonary tumors, so that false interpretation of PET image data sets can occur.

### SUMMARY

At least one embodiment of the present invention provides a method and/or a hybrid imaging modality with which generally better differentiation of areas of increased signal intensity in PET image data sets can be achieved.

At least one embodiment of the invention is directed to a method for producing a combination image. Advantageous further developments of the invention are set forth in the dependent claims.

According to at least one embodiment of the invention, a PET image data set and a perfusion image data set are acquired. These need neither represent the same region of interest nor have identical resolutions, slice thicknesses or the like. The image data sets only need to overlap in the represented part of the object under examination in which combining of the respective information is deemed necessary. Registration of the image data sets must also be possible so that the image data sets can be combined in a meaningful manner.

At least one embodiment of the invention is directed to a hybrid imaging modality. This comprises a PET scanner and at least one second imaging modality, in particular an MRI scanner and/or a CT scanner, as well as a control device designed to carry out embodiments of the method.

Embodiments of the above mentioned method can be implemented in the control device as software or even as (hard-wired) hardware.

The advantageous embodiments of the method according to the invention correspond to corresponding embodiments of the hybrid imaging modality according to the invention. To avoid unnecessary repetitions, reference will therefore be made to the corresponding method features and the advantages thereof.

### BRIEF DESCRIPTION OF THE DRAWINGS

Further advantages, features and specific characteristics of the present invention will emerge from the following description of advantageous embodiments of the invention.

In the accompanying drawings

FIG. 1 shows a hybrid imaging modality according to an embodiment of the invention,

FIG. 2 shows a flow chart of the method according to an embodiment of the invention,

FIG. 3 shows a combination image in a first embodiment,

FIG. 4 shows a combination image in a second embodiment, and

FIG. 5 shows a combination image in a third embodiment.

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

According to at least one embodiment of the invention, a PET image data set and a perfusion image data set are acquired. These need neither represent the same region of interest nor have identical resolutions, slice thicknesses or the like. The image data sets only need to overlap in the represented part of the object under examination in which combining of the respective information is deemed necessary. Registration of the image data sets must also be possible so that the image data sets can be combined in a meaningful manner.

Similarly to the acquiring of the PET image data set, acquisition of a perfusion image data is to be understood as meaning all the steps resulting in spatially resolved perfusion information or rather an image representing it. The signals of one or more spatially resolved “raw” image data sets can be further processed or combined as required. It is merely essential to obtain spatially resolved perfusion information.

This does not need to be absolutely quantified, i.e. the perfusion image data set does not need to show that the perfusion is 4 ml/(g*min) in a particular image element. It is sufficient if a relative quantification can be determined, i.e. if twice the signal intensity of one image element, represented by a corresponding numerical value, corresponds to twice the perfusion in another image element.

It is self-evidently assumed here that the image data sets are made up of image elements, i.e. pixels or voxels, each image element being assigned a numerical value corresponding to a recorded signal intensity. The image data sets can be stored as matrices, arrays or in some other form. For visualization, each numerical value is assigned a grayscale value or color value depending on the color palette used. This can be displayed at the image edge and indicates which color is assigned to which numerical value or value range. It is usual here to provided a grayscale palette and a color palette.

The grayscale palette is normally designed such that the color black is assigned to the lowest numerical value represented in an image and the color white to the highest.

Although a large number of color palettes exist, the “Rainbow” palette in “IDL” software is widely used. Here the color “black” constitutes the lowest numerical value and the color “red” the highest. The palette extends from “black” through the colors “purple”, “dark blue”, “light blue”, “light green”, “green”, “yellow” and finally “red”, wherein each numerical value of an image element is assigned a color for visualization and an image is displayed. Likewise well known is the “Matlab” programming language which provides the “Jet” color palette. This only differs from the “Rainbow” color palette in that “dark blue” is assigned to the lowest numerical value instead of “black”. The further sequence may still differ in the precise gradation or rather the number of intermediate steps, but is otherwise identical to the color sequence described above.

In any case, a control device is provided with at least one color palette with which the user of the corresponding control device is familiar. In a step, it is inventively provided that the color representation of the perfusion image data set is inverted. To be more precise, the assignment of the colors of a color palette to numerical values, in particular the numerical values occurring in the perfusion image data set, is reversed or inverted. This can take place in three different ways.

First of all, the reciprocals can be determined for each numerical value of the image elements of the perfusion image data set according to the formula

reciprocal=1/numerical value.

These reciprocals are then each assigned a color value of the color palette, thus producing the perfusion image data set. However, this results in distortions, since although the spacing of the numbers one, two and three is identical, that of the reciprocals of one, ½ and ⅓ is not.

In another embodiment, a numerical value is determined for each image element according to the formula

numerical value_new=numerical value_max−numerical value_old.

Numerical value_max is the highest numerical value of the numerical values of all the image elements of the perfusion image data set, which means that what was originally the largest numerical value becomes zero and also becomes the smallest numerical value. All other new numerical values calculated are greater than zero and are between zero and the numerical value determined from what was originally the smallest numerical value. In principle, any other numerical value, even one which does not occur in the perfusion image data set, can also be used as numerical value_max. The lowest numerical value is then no longer zero, but greater or less than zero, thereby enabling “offsets” to be produced.

Alternatively, the color palette itself can also be inverted, i.e. the lowest numerical value is assigned the color “red”, etc. The lowest numerical value is therefore assigned the color which is otherwise assigned to the highest numerical value and vice versa.

Inversion of the color palettes is self-evidently not restricted to the color palettes described, but is similar for any other color palettes.

Alternatively or additionally to inversion of the color or grayscale palettes, a threshold value can be predefined and only the regions or rather image elements of the perfusion image data set that are below the threshold value are used to create the combination image. For each image element, the numerical value is self-evidently compared with the threshold value and retained or discarded. This makes it immediately apparent which signal areas of the PET image data set are in regions of low perfusion and which are not. Low perfusion here means perfusion below the threshold value.

For absolute quantification of perfusion, the threshold value can be predefined as a numerical value. For only relative quantification, it can be specified as a percentage of the maximum value of all the numerical values of the image elements of the perfusion image data set, or of the average value.

After inversion of the perfusion image data set or extraction of the areas of the perfusion image data set below the threshold value, the perfusion image data set and the PET image data set are superimposed to produce a combination image, with the perfusion image data set in the background and PET image data set in the foreground.

In this way, the areas with increased signal intensity in the PET image data set that are in areas of lower perfusion are highlighted with warning colors.

The PET image data set is preferably assigned a color palette which is different from that of the perfusion image data set. This means that the regions of the combination image that were derived from the PET image data set are more easily identifiable and better distinguishable from those of the perfusion image data set.

In one embodiment, an MRI scanner is used as a second imaging modality. Magnetic resonance tomography is a well-known imaging method for visualizing anatomical structures as well as functional variables. In particular, it is known to use magnetic resonance tomography to generate perfusion image data sets.

In a further development of the invention, an arterial spin labeling (ASL) method can be used to acquire the perfusion image data set by means of magnetic resonance tomography. A slice upstream of the slice of interest or rather of the imaging volume is prepared. This preparation is also known as spin tagging or spin labeling. In the slice of interest, the prepared spins perfuse into the tissue and change the signal intensity of the perfused region. The signal changes are therefore indicative of perfusion. To determine the signal change, a reference image must be taken in which the preparation is omitted, the imaging conditions remaining otherwise unchanged.

There are two different types of ASL, namely pulsed arterial spin labeling (PASL) and continuous arterial spin labeling (CASL). These differ merely in the way in which the signal is prepared and both can be used. The spins thus prepared are sometimes also termed endogenous tracers, whereas administered contrast agents are known as exogenous tracers. In the present application, the term contrast agent is used to denote a substance to be administered which changes the contrast behavior in an image when it is present in the imaged region of the object under examination.

In an alternative embodiment, the perfusion image data set can be acquired using a contrast agent. The latter is usually administered near the site of interest and then transported thereinto. With continuous or selective imaging of the region of interest prior to, during and some time after administration, particularly during the arterial phase of contrast agent wash-in, the contrast agent bolus in the tissues and the contrast agent uptake into the tissue can be visualized and spatially resolved perfusion information calculated.

If a contrast agent is used to produce the perfusion image data set, it is possible to deploy additional imaging modalities. In particular, an X-ray machine or a CT scanner or an ultrasound unit can be used. Preferably a CT scanner is used, because there are already hybrid imaging modalities comprising a PET scanner and a CT scanner, and CT scanners are known which also enable simple radiographic images to be produced. Because of the repeated recording of individual image data sets to establish the perfusion image data set, it is desirable to obtain a high temporal resolution while minimizing the radiation load. This is possible by taking radiographic images.

Preferably a contrast agent can be used which also serves as a radionuclide or radiopharmaceutical for acquiring the PET image data set. The contrast agent must be tailored to the imaging modality. In the case of an MRI scanner, for example, a gadolinium or manganese chelate additionally incorporating a radionuclide could be used. In magnetic resonance tomography, so-called intelligent contrast agents are also known which contain a part resulting in concentration of the contrast agent on the basis of metabolic processes or other processes. These contrast agents are also known as event or molecule markers. A radionuclide can also be incorporated in intelligent contrast agents of this kind to produce a radiopharmaceutical.

This makes it possible for the PET image data set and the perfusion image data set to be co-registered on the basis of the temporal and/or spatial signal response of the contrast agent or rather radionuclide. The contrast agent can be administered a plurality of times, but a single administration may also suffice. The perfusion image data set must then be created first, e.g. by monitoring the uptake and perfusion of the contrast agent. If the radionuclide is embedded in a radiopharmaceutical, metabolization will require some time anyway. This is used to create the perfusion image data set. In particular, due to the possibility of a single administration of a contrast agent being sufficient for obtaining both the perfusion image data set and the PET image data set, the use of such a contrast agent is of interest, as only a limited (contrast agent) volume can be introduced into a patient's vascular system.

Because of the simultaneous accumulation of the contrast agent and radionuclide, areas of increased signal intensity are produced in the PET image data set which correlate with areas of increased or reduced signal intensity in the perfusion image data set in respect of size, spacing and number. This enables the perfusion image data set and the PET image data set to be co-registered without additional aids.

If a hybrid imaging modality is used, registration is intrinsically provided. The image data sets of the second imaging modality, and also those for creating the perfusion image data set, can then be used to perform motion correction on the signals acquired using the PET scanner. Self-evidently, anatomical image data sets of the region of interest can also be acquired using the second imaging modality during operation of the PET scanner. By comparing a plurality of anatomical image data sets, motion information is obtained which can be taken into account during calculation of a PET image data set.

At least one embodiment of the invention is directed to a hybrid imaging modality. This comprises a PET scanner and at least one second imaging modality, in particular an MRI scanner and/or a CT scanner, as well as a control device designed to carry out embodiments of the method.

Embodiments of the above mentioned method can be implemented in the control device as software or even as (hard-wired) hardware.

The advantageous embodiments of the method according to the invention correspond to corresponding embodiments of the hybrid imaging modality according to the invention. To avoid unnecessary repetitions, reference will therefore be made to the corresponding method features and the advantages thereof.

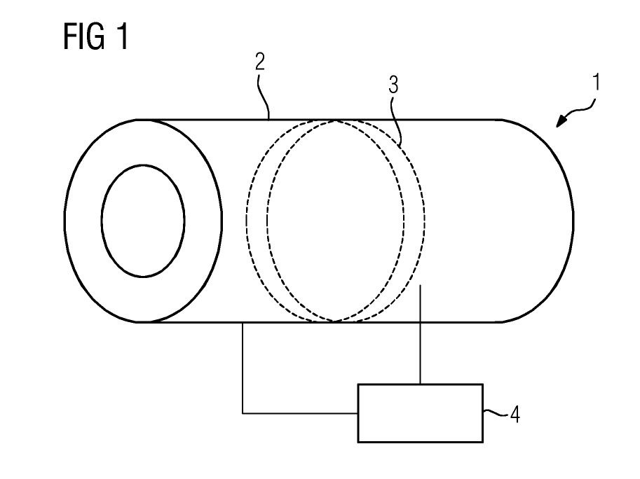

FIG. 1 shows a hybrid imaging modality 1 comprising an MRI scanner 2 having a detector ring 3 disposed therein, and a control unit 4. For the sake of clarity, the many individual components of the MRI scanner 2, such as gradient coils, excitation and detection coil, patient table, and also those of the PET scanner, are not shown.

The detector ring 3 of the PET scanner is in the homogeneous region of the main magnetic field of the MRI scanner 2, so that simultaneous scans using both imaging modalities are possible. The excitation coil, or “body coil” as it is also known, can form a unit with the detector ring in order not to unnecessarily limit the space for the patient.

This rigid design also provides registration of the images taken using the different imaging modalities.

The method described is realized in software in the control unit 4. In particular, it can be carried out “at the press of a button” after positioning of the patient and administration of a radiopharmaceutical or more specifically a contrast agent.

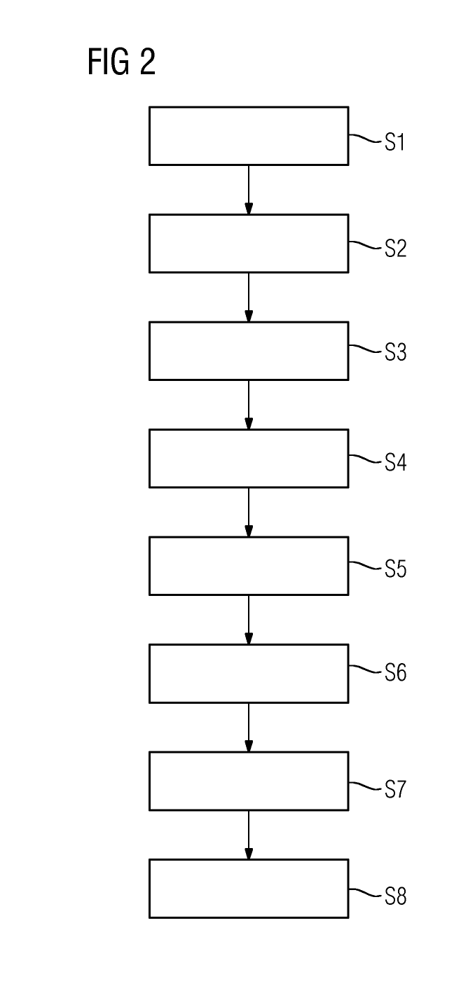

FIG. 2 shows a flow chart for the taking of a combination image. In step S1, a patient is moved into the hybrid imaging modality 1, positioned, and the magnetic field is homogenized. In step S2, a radiopharmaceutical, preferably 18F-FDG, is administered. A reference image is then acquired using the MRI scanner (step S3). Only then is an MR contrast agent administered, preferably a contrast agent containing gadolinium (step S4). Immediately after administration of the MR contrast agent, overviews are continuously taken several seconds apart, e.g. at 10-second intervals. These are identical to the reference image and to one another apart from the time of acquisition. To calculate a perfusion image data set it basically suffices to take a single overview. In step S5, the perfusion image data set is calculated from the reference image and the overview by subtracting the overview image element by image element from the reference image. The other overviews are used for motion correction of the signals measured using the detector ring 3.

Acquisition of the annihilation events is carried out until at least one PET image data set can be calculated from the scan data as step S6.

In step S7, the color palette of the perfusion image data set is inverted by mirroring the color assignment. In addition, a threshold value is applied to the perfusion image data set, all the image elements above said threshold value being discarded.

Then, in step S8, the PET image data set and the perfusion image data set are superimposed to form a combination image, the perfusion image data set being in the background. All the signal areas in the PET image data set due to the accumulations of the radiopharmaceutical, which are in regions of reduced perfusion, can thus be visually observed immediately.

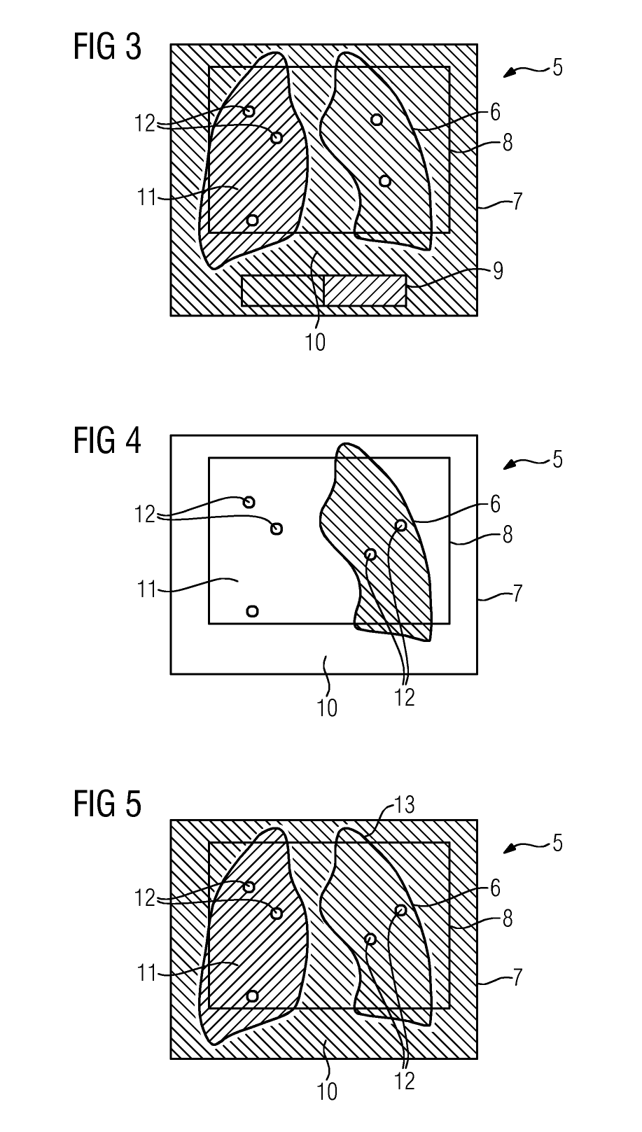

FIG. 3 shows a combination image 5 of a lung 6. The perfusion image data set 7 is in the background and the PET image data set 8 in the foreground. On one side of the combination image a color scale 9 is disposed which indicates the color palette used. The color palette is inverted compared to other representations.

For reasons of representability, the perfusion image data set 7 is shown in two sections. In region 10, the perfusion is higher, in region 11 lower. Some of the signal areas 12 of the PET image data set 8 are within region 10 and some within region 11.

If it is assumed that the reduced perfusion in the lung 6 in region 11 is due to a pulmonary embolism, this information can also be taken into account for assessing the signal areas 12.

FIG. 4 shows an alternative representation of a combination image 5. Only region 11 of the perfusion image data set 7 is shown. Some of the signal areas 12 of the PET image data set 8 are therefore inside region 11 and some outside. The perfusion image data set 7 does not map the entire region of interest, as the combination image 5 was created using a threshold value by means of which all the image elements having a numerical value above the threshold value were filtered out. As a result, the region 10 is suppressed.

FIG. 5 shows another alternative representation of a combination image 5. Both regions 10 and 11 of the perfusion image data set 7 are shown. To separate these regions, a clearly visible dividing line 13 is indicated.

The method according to an embodiment of the invention and the MRI scanner according to an embodiment of the invention are self-evidently closely interrelated, and features of the invention that have been described as method aspects also essentially apply to the MRI scanner. The converse applies to features described in respect of the MRI scanner which can also be relevant to the method.

It is also self-evident that features described with reference to individual embodiments can also be implemented for other embodiments or designs, unless expressly described otherwise or out of the question for technical reasons.

What is claimed is:

1. A method for producing a combination image, comprising:
acquiring at least one PET image data set depicting a region of interest;
acquiring at least one perfusion image data set depicting the region of interest using a second imaging modality;
establishing a threshold value for the at least one acquired perfusion image data set and at least one of selecting areas of the perfusion image data set that are below the threshold value and inverting a color palette or grayscale palette of the at least one perfusion image data set; and
combining the acquired at least one PET image data set and the at least one perfusion image data set to form the combination image.

2. The method of claim 1, wherein an MRI (magnetic resonance imaging) scanner is used as the second imaging modality.

3. The method of claim 2, wherein an arterial spin labeling method is used to acquire the at least one perfusion image data set.

4. The method of claim 1, wherein a CT (computed tomography) scanner is used as the second imaging modality.

5. The method of claim 4, wherein the at least one perfusion image data set is acquired using a contrast agent.

6. The method of claim 5, wherein the contrast agent one employed both to obtain the at least one PET image data set and as a radiotracer.

7. The method of claim 6, wherein the at least one PET image data set and the at least one perfusion image data set are co-registered on the basis of the temporal and spatial signal response of the contrast agent.

8. The method of claim 1, wherein the threshold value is predefined as a function of the maximum value of the at least one perfusion image data set or as a fixed value.

9. The method of claim 1, wherein anatomical image data sets are acquired using the second imaging modality which allow motion correction of the positron emission signals.

10. A hybrid imaging modality for forming a combination image, comprising:
a PET scanner;
at least one second imaging modality; and
a control device configured to
acquire at least one PET image data set depicting a region of interest via the PET scanner,
acquire at least one perfusion image data set depicting the region of interest via the second imaging modality,
establish a threshold value for the at least one acquired perfusion image data set and at least one of selecting areas of the perfusion image data set that are below the threshold value and inverting a color palette or grayscale palette of the at least one perfusion image data set, and
combine the acquired at least one PET image data set and the at least one perfusion image data set to form the combination image.

11. The method of claim 1, wherein the region of interest is at least part of a lung.

12. The method of claim 1, wherein the at least one perfusion image data set is acquired using a contrast agent.

13. The method of claim 12, wherein the contrast agent one employed both to obtain the at least one PET image data set and as a radiotracer.

14. The method of claim 13, wherein the at least one PET image data set and the at least one perfusion image data set are co-registered on the basis of the temporal and spatial signal response of the contrast agent.

15. The method of claim 2, wherein anatomical image data sets are acquired using the second imaging modality which allow motion correction of the positron emission signals.

16. The method of claim 4, wherein anatomical image data sets are acquired using the second imaging modality which allow motion correction of the positron emission signals.

17. The hybrid imaging modality of claim 10, wherein an MRI (magnetic resonance imaging) scanner is used as the second imaging modality.

18. The hybrid imaging modality of claim 10, wherein a CT (computed tomography) scanner is used as the second imaging modality.
